(19) United States
(12) Patent Application Publication
(10) Pub. No.: US 2026/0335023 A1
(43) Pub. Date: Oct. 29, 2026

(54) MEDICAL IMAGING SYSTEM FOR IDENTIFICATION OF DYNAMIC ELECTRODE ASSEMBLY ELEMENTS

(71) Applicant: Boston Scientific Scimed, Inc., Maple Grove, MN (US)

(72) Inventors: Caryn Clark, Medford, MA (US); Charlene Leung, Scarborough (CA); Michelle Elizabeth Lustrino, Watertown, MA (US); Tobias Schroeder, Bedford, MA (US); Laksh Deepak Raura, Woburn, MA (US)

(21) Appl. No.: 19/656,587

(22) Filed: Apr. 23, 2026

Related U.S. Application Data
(60) Provisional application No. 63/793,361, filed on Apr. 23, 2025.
(60) Provisional application No. 63/892,609, filed on Oct. 2, 2025.

(51) Int. Cl. A61B 34/20; A61B 8/00; A61B 8/08; A61B 8/12; A61B 18/00; A61B 18/14

(52) U.S. Cl. CPC A61B 34/20; A61B 8/0841; A61B 8/0883; A61B 8/12; A61B 8/463; A61B 8/54; A61B 18/1492; A61B 2018/00577; A61B 2018/00613; A61B 2034/2063

(57) ABSTRACT

A medical imaging system includes a therapeutic, mapping or diagnostic catheter having a dynamic electrode assembly with elements, an ultrasound imaging catheter and a controller. The dynamic electrode assembly is transitionable from a collapsed configuration to an expanded configuration. The ultrasound imaging catheter images the therapeutic, mapping or diagnostic catheter within a patient. The controller generates a visualization for facilitation on a display. The visualization includes an ultrasound image having a representation of an element of the electrode assembly based on signals provided by the ultrasound imaging catheter. The controller identifies the element of the electrode assembly from the representation of the element in the ultrasound image and position and orientation data obtained from the therapeutic, mapping or diagnostic catheter. The visualization further includes a visual indicator associated with the representation of the element based on the identified element of the electrode assembly.

### CROSS REFERENCE TO RELATED APPLICATIONS

The present application claims priority to U.S. Provisional Application No. 63/793,361 entitled “MEDICAL IMAGING SYSTEM FOR IDENTIFICATION OF DYNAMIC ELECTRODE ASSEMBLY ELEMENTS,” filed Apr. 23, 2025, and U.S. Provisional Application No. 63/892,609 entitled “IDENTIFICATION OF INDIVIDUAL DYNAMIC ELEMENTS ON 2D ICE,” filed Oct. 2, 2025, which are hereby incorporated by reference in their entirety.

### TECHNICAL FIELD

The present disclosure relates to medical systems and methods for facilitating visualization of tissue from within a patient. More specifically, the present disclosure relates to medical systems, catheters, and methods implementing ultrasonic transducer devices.

### BACKGROUND

Intracardiac echocardiography (ICE) is an ultrasonic imaging modality that has become a common aspect of several percutaneous interventional and electrophysiology procedures. ICE can provide high resolution and real-time visualizations of cardiac structures, continuous monitoring of catheter location within the heart, and early recognition of procedural complications such as pericardial effusion or thrombus formation. An ICE catheter is typically inserted in a vein, such as via standard femoral venous introducer into the femoral vein, and then passed from the vein into the heart alongside other tools. Another common ultrasonic imaging modality includes transesophageal echocardiography (TEE) and fluoroscopy to image and visualize tools within the heart, which requires an anesthesiologist and additional echocardiologist to keep the patient under general anesthesia and to manipulate an ultrasonic probe within the airway. In contrast to TEE, ICE can be performed by the primary operator of the interventional procedure under conscious sedation, without endotracheal intubation, and with a reduced risk of esophageal trauma. Additionally, ICE reduces fluoroscopy exposure for both the patient and the operator. For these reasons, ICE is a preferred imaging modality in certain procedures such as atrial septal defect closure and catheter ablation of cardiac arrhythmias and includes an emerging role in other procedures such as mitral valvuloplasty, transcatheter aortic valve replacement, and left atrial appendage closure.

Two forms of ICE are available. Radial or rotational ICE uses a single piezoelectric crystal mounted on a tip of a six to ten French (Fr.) catheter. A rotating transducer provides cross-sectional images in a radial plane perpendicular to a longitudinal axis of the catheter. Rotational ICE operates at imaging frequencies that are useful for near-field imaging of up to six or eight centimeters but is limited for far-field imaging. Phased-array ICE uses a multi-element transducer, such as a sixty-four-element transducer, mounted on a distal end of an eight to ten Fr. steerable catheter that can often be deflected in four directions including anterior, posterior, right, and left. The device provides a wedge-shaped image, such as a ninety-degree sector plane, from the side of the catheter that can be displayed on a conventional ultrasound workstation. When contrasted with mechanical rotational ICE systems, phased-array ICE can provide for greater image-depth penetration (up to 15 cm), greater maneuverability, and the ability to acquire Doppler and color flow imaging.

### SUMMARY

In Example 1, a medical imaging system, comprising: a catheter including a dynamic electrode assembly having a plurality of elements, the dynamic electrode assembly transitionable from a collapsed configuration to an expanded configuration; an ultrasound imaging catheter configured to image the catheter within a patient; and a controller configured to: generate a visualization for facilitation on a display, the visualization including an ultrasound image having a representation of an element of the electrode assembly based on signals provided by the ultrasound imaging catheter; and identify the element of the electrode assembly from the representation of the element in the ultrasound image and position and orientation data obtained from the catheter; wherein the visualization further includes a visual indicator associated with the representation of the element based on the identified element of the electrode assembly.

In Example 2, the medical imaging system of Example 1, wherein the plurality of elements includes a plurality of splines and a plurality of electrodes.

In Example 3, the medical imaging system of Example 2, wherein the plurality of electrodes includes a plurality of ablation electrodes.

In Example 4, the medical imaging system of any of Examples 1-3, wherein the visual indicator is an overlay on the ultrasound image.

In Example 5, the medical imaging system of Example 4, wherein the visual indicator is a direct overlay on the ultrasound image.

In Example 6, the medical imaging system of Example 5, wherein the direct overlay includes a delineation of the of at least one of a spline and an electrode.

In Example 7, the medical imaging system of Example 4, wherein the visual indicator is an indirect overlay.

In Example 8, the medical imaging system of any of Examples 1-7, wherein the controller configured to identify the element based on the three-dimensional position of the catheter with respect to tissue as provided in an anatomical map.

In Example 9, the medical imaging system of Example 8, wherein the controller configured to identify the element is based on a detected shape of the electrode assembly.

In Example 10, the medical imaging system of Example 9, wherein the detection of shape is based on impedance measurements of electrodes on the electrode assembly.

In Example 11, the medical imaging system of any of Examples 1-10, wherein the controller is configured to identify the element based on image processing techniques.

In Example 12, the medical imaging system of any of Examples 1-10, wherein the visual indicator indicates whether the identified element is within a predetermined proximity to tissue.

In Example 13, the medical imaging system of Example 12, wherein the visual indicator indicates whether the identified element is in contact with the tissue.

In Example 14, the medical imaging system of any of Examples 1-13, wherein the visualization includes a corresponding visual indicator in a representation of the element in a graphical representation generated via an electroanatomical mapping system.

In Example 15, the medical imaging system of any of Examples 1-14, wherein the catheter is an electroporation catheter and the ultrasound imaging catheter is an intracardiac echocardiography (ICE) catheter.

In Example 16, a medical imaging system, comprising: a therapeutic, mapping or diagnostic catheter including a dynamic electrode assembly having a plurality of elements, the dynamic electrode assembly transitionable from a collapsed configuration to an expanded configuration; an ultrasound imaging catheter configured to image the therapeutic, mapping or diagnostic catheter within a patient; and a controller configured to: generate a visualization for facilitation on a display, the visualization including an ultrasound image having a representation of an element of the electrode assembly based on signals provided by the ultrasound imaging catheter; and identify the element of the electrode assembly from the representation of the element in the ultrasound image and position and orientation data obtained from the therapeutic, mapping or diagnostic catheter; wherein the visualization further includes a visual indicator associated with the representation of the element based on the identified element of the electrode assembly.

In Example 17, the medical imaging system of Example 16, wherein the plurality of elements includes a plurality of splines and a plurality of electrodes.

In Example 18, the medical imaging system of Example 17, wherein the plurality of electrodes includes a plurality of ablation electrodes.

In Example 19, the medical imaging system of Example 16, wherein the visual indicator is an overlay on the ultrasound image.

In Example 20, the medical imaging system of Example 19, wherein the visual indicator is a direct overlay on the ultrasound image.

In Example 21, the medical imaging system of Example 20, wherein the direct overlay includes a delineation of the of at least one of a spline and an electrode.

In Example 22, the medical imaging system of Example 19, wherein the visual indicator is an indirect overlay.

In Example 23, the medical imaging system of Example 22, wherein the indirect overlay is an inset.

In Example 24, the medical imaging system of Example 16, wherein the controller configured to identify the element based on the three-dimensional position of the therapeutic, mapping or diagnostic catheter with respect to tissue as provided in an anatomical map.

In Example 25, the medical imaging system of Example 24, wherein the controller configured to identify the element is based on a detected shape of the electrode assembly.

In Example 26, medical imaging system of claim 16, wherein the visualization includes a corresponding visual indicator in a representation of the element in a graphical representation generated via an electroanatomical mapping system.

In Example 27, the medical imaging system of Example 16, wherein the visual indicator indicates whether the identified element is within a predetermined proximity to tissue.

In Example 28, the medical imaging system of Example 27, wherein the visual indicator indicates whether the identified element is in contact with the tissue.

In Example 29, the medical imaging system of Example 16, wherein the therapeutic, mapping or diagnostic catheter is an electroporation catheter and the ultrasound imaging catheter is an intracardiac echocardiography (ICE) catheter.

In Example 30, a medical imaging controller, comprising: a memory to store a set of instructions; and a processor to execute the set of instructions to: generate a visualization having an ultrasound image of a therapeutic, mapping or diagnostic catheter having an electrode assembly having a plurality of elements, wherein the ultrasound image includes a two-dimensional imaging plane having a representation of an element of the electrode assembly; identify from the ultrasound image which element of the electrode assembly corresponds with the representation of the element of the electrode assembly based on position and orientation data obtained from the therapeutic, mapping or diagnostic catheter; and generate a visual indicator in the visualization, the visual indicator associated with the identified element.

In Example 31, the medical imaging controller of Example 30, wherein the visual indicator indicates whether the identified element is within a predetermined proximity to tissue.

In Example 32, The medical imaging controller of Example 31, wherein the visual indicator indicates whether the identified element is in contact with the tissue.

In Example 33, the medical imaging controller of Example 30, wherein the controller configured to identify from the ultrasound image is based on a three-dimensional position of the ablation catheter with respect to tissue as provided in the anatomical map.

In Example 34, a method of generating an image for facilitation on a display, the method comprising: generating a visualization having an ultrasound image of a therapeutic, mapping or diagnostic catheter having an electrode assembly having a plurality of elements, wherein the ultrasound image includes a two-dimensional imaging plane having a representation of an element of the electrode assembly; identifying from the ultrasound image which element of the electrode assembly corresponds with the representation of the element of the electrode assembly base on position and orientation data obtained from the therapeutic, mapping or diagnostic catheter; and generating a visual indicator in the visualization, the visual indicator associated with the identified element.

In Example 35, the method of Example 34 further comprising assigning an identifier to each element of the plurality of elements of the electrode assembly, and determining, based on the ultrasound image, which identifier corresponds with the representation of the element of the electrode assembly wherein the visual indicator is associated with the assigned identifier.

While multiple embodiments are disclosed, still other embodiments of the present disclosure will become apparent to those skilled in the art from the following detailed description, which shows and describes illustrative embodiments of the disclosure. Accordingly, the drawings and detailed description are to be regarded as illustrative in nature and not restrictive.

### BRIEF DESCRIPTION OF THE DRAWINGS

FIG. 1 is a diagram illustrating an exemplary clinical setting for treating a patient, and for treating a heart of the patient, using an electrophysiology system and a medical imaging system.

FIG. 2 is a side view of a catheter suitable for use in the electrophysiological system of FIG. 1, in which the catheter is in a collapsed configuration.

FIG. 2A is a side view of a distal end of the catheter of FIG. 2A in the collapsed configuration.

FIG. 2B is a perspective view of the distal end of the catheter of FIG. 2A in an intermediate configuration.

FIG. 2C is a perspective view of the distal end of the catheter of FIG. 2A in an expanded configuration.

FIG. 3 is a schematic diagram a medical imaging system of the exemplary clinical setting of FIG. 1.

FIG. 4 is a block diagram illustrating an embodiment of a controller for use with the example electrophysiology system of FIG. 1.

FIG. 5 is a block diagram illustrating an embodiment of a configuration of the controller of FIG. 4.

FIG. 5A is a schematic diagram illustrating an implementation of a feature of the configuration of FIG. 5.

FIG. 6 is a schematic screen shot of an embodiment of a visualization generated with the configuration of the controller of FIG. 5.

FIG. 6A is a schematic screen shot of another embodiment of a visualization generated with the configuration of the controller of FIG. 5.

FIG. 6B is a schematic screen shot of another embodiment of a visualization generated with the configuration of the controller of FIG. 5.

FIG. 6C is a schematic screen shot of another embodiment of a visualization generated with the configuration of the controller of FIG. 5.

FIG. 7 is a schematic screen shot of another embodiment of a visualization generated with the configuration of the controller of FIG. 5.

While the disclosure is amenable to various modifications and alternative forms, specific embodiments have been shown by way of example in the drawings and are described in detail below. The intention, however, is not to limit the disclosure to the particular embodiments described. On the contrary, the disclosure is intended to cover all modifications, equivalents, and alternatives falling within the scope of the disclosure as defined by the appended claims.

### DETAILED DESCRIPTION

For purposes of promoting an understanding of the principles of the present disclosure, reference is now made to the examples illustrated in the drawings, which are described below. The illustrated examples disclosed herein are not intended to be exhaustive or to limit the disclosure to the precise form disclosed in the following detailed description. Rather, these exemplary embodiments were chosen and described so that others skilled in the art may use their teachings. It is not beyond the scope of this disclosure to have a number (e.g., all) the features in a given example used across all examples. Thus, no one figure should be interpreted as having any dependency or requirement related to any single component or combination of components illustrated therein. Additionally, various components depicted in a given figure may be, in examples, integrated with various ones of the other components depicted therein (and/or components not illustrated), all of which are considered to be within the ambit of the present disclosure.

Ablation procedures are used to treat many different conditions in patients. Ablation can be used to treat cardiac arrhythmias, benign tumors, cancerous tumors, and to control bleeding during surgery. Usually, ablation is accomplished through thermal ablation techniques including radio-frequency (RF) ablation and cryoablation. In RF ablation, a probe is inserted into the patient and radio frequency waves are transmitted through the probe to the surrounding tissue. The radio frequency waves generate heat, which destroys surrounding tissue and cauterizes blood vessels. In cryoablation, a hollow needle or cryoprobe is inserted into the patient and cold, thermally conductive fluid is circulated through the probe to freeze and kill the surrounding tissue. RF ablation and cryoablation techniques indiscriminately kill tissue through cell necrosis, which may damage or kill otherwise healthy tissue, such as tissue in the esophagus, phrenic nerve cells, and tissue in the coronary arteries.

Another ablation technique uses electroporation. In electroporation, or electro-permeabilization, an electrical field is applied to cells to increase the permeability of the cell membrane. The electroporation can be reversible or irreversible, depending on the strength of the electric field. If the electroporation is reversible, the increased permeability of the cell membrane can be used to introduce chemicals, drugs, and/or deoxyribonucleic acid (DNA) into the cell, prior to the cell healing and recovering. If the electroporation is irreversible, the affected cells are killed through apoptosis. Irreversible electroporation can be used as a nonthermal ablation technique. In irreversible electroporation, trains of short, high voltage pulses are used to generate electric fields that are strong enough to kill cells through apoptosis. In ablation of cardiac tissue, irreversible electroporation can be a safe and effective alternative to the indiscriminate killing of thermal ablation techniques, such as RF ablation and cryoablation. Irreversible electroporation can be used to kill targeted tissue, such as myocardium tissue, by using an electric field strength and duration that kills the targeted tissue but does not permanently damage other cells or tissue, such as non-targeted myocardium tissue, red blood cells, vascular smooth muscle tissue, endothelium tissue, and nerve cells.

Electrophysiological procedures, which include catheter ablation to treat a variety of heart conditions such as supraventricular and ventricular arrhythmia, involve a visualization of the heart and heart activity followed by treatment of identified target tissue. Electroanatomical mapping is a visualization technique that allows a clinician to accurately determine the location of an arrhythmia, define cardiac geometry in three dimensions, delineate areas of anatomic interest, and permits imaging of the catheter assembly for positioning and manipulation. For instance, electroanatomical mapping involves the mapping of electrical activity in the heart based on cardiac signals, such as at various locations on the endocardium surface, to identify the site of origin of the arrhythmia followed by a targeted ablation of the site. Once the target ablation site has been identified, the clinician will access the target tissue, under visualization to perform the ablation with an ablation catheter. An example of an ablation catheter includes an elongated shaft having a distal portion with an expandable electrode assembly. In examples, the electrode assembly includes a set of splines and electrodes on the splines to perform the ablation. The electrode assembly is deployed in an expanded configuration and maneuvered proximate the target tissue to perform the ablation of anatomic interest.

Clinicians often use an ultrasound image as the visualization during catheter ablation. For example, clinicians often use an ICE catheter to generate a field of view on a display to verify the position of the ablation catheter with respect to the target tissue and to verify the electrode assembly is in contact with tissue. Clinicians attempt to identify the splines and electrodes of the electrode assembly and determine whether the electrodes are in contact with target tissue on, typically, a wedge-shaped two-dimensional ultrasound image, such as a ninety-degree sector plane. Interpreting the positioning of the three-dimensional electrode array in the two-dimensional ultrasound imaging plane can be difficult. In some examples, the resolution of the ultrasound images, such as in B-mode, results in images of the anatomy that are not well distinguishable. In some examples, a clinician may have difficulty reconciling the visualizations and data on an electroanatomic map, such as electrical signal data and electrical impedance data of the electrodes, with the ultrasound image. In other examples, a clinician may be unclear as to the location the imaging plane of the ICE catheter with respect to the ablation catheter. Uncertainties in interpreting the ultrasound image can lead to reduce confidence in the placement of the catheter and drawn-out procedures.

The present disclosure is directed toward a medical imaging system to include a visual indicator or visual indicators, such as in an overlay, with an ultrasound image to provide information regarding the electrode assembly of the ablation catheter in the ultrasound image. The medical imaging system is configured to generate a visualization including an ultrasound image, such as image of the anatomy in brightness mode, or B-mode. The medical imaging system is also configured to generate a visual indicator that provides information regarding the electrode assembly of the ablation catheter in the ultrasound image as an overlay of the ultrasound image. In one embodiment, the visual indicator includes an electrode assembly element identifier that identifies elements of the electrode assembly, such as splines or electrodes, in view on the ultrasound image. In one embodiment, the visual indicator includes a contact visual identifier that indicates whether a spline or electrode in the ultrasound image is in contact with tissue.

The visual indicators help the clinician identify features of the electrode assembly, such as splines or electrodes, on the ultrasound image. For instance, the splines is highlighted on the ultrasound image or identified by color on the ultrasound image in which each spline of the electrode assembly is assigned a color from a set of colors. In one embodiment, the visual indicator is a direct overlay on the ultrasound image, such that the representation of the spline in the ultrasound image is highlighted or colored. In one embodiment, features of the electrode assembly are assigned an individual visual indicator. For instance, each spline is assigned a color such as a color of the spline used in the electroanatomical mapping system. In another embodiment, the visual indicator is an indirect indication, such as an indication not made to the representation of the spline in the ultrasound image. Rather, a text notification that a given spline or splines are in plane. When the electrode assembly is detected in the ultrasound image, a user has the option to generate an overlay on the ultrasound image to generate the visual indicator to the features of the electrode assembly. For instance, a clinician may be unclear from a B-mode ultrasound image as to which spline from the electrode assembly is in the two-dimensional imaging plane. Activation of the identification visual indicators can generate an overlay on the ultrasound image to apply the assigned visual indicator to the spline. A clinician can then detect from the overlay on the ultrasound image as to which spline or splines are in the imaging plane.

In one embodiment, the visual indicator to the ultrasound image provides an indication as to whether the electrode assembly, such as the splines or electrodes, in view on the ultrasound image are in contact with tissue. For instance, a clinician may be unclear from a B-mode ultrasound image as to whether a spline or electrode in the two-dimensional imaging plane is in contact with tissue. Activation of the tissue contact visual indicators can generate an overlay on the ultrasound image to indicate that the element of the electrode assembly is in contact with tissue. In embodiments in which the tissue contact visual indicators are applied with the identification visual indicators, the clinician can then detect from the overlay on the ultrasound image as to which spline or splines are in the imaging plane and more clearly determine whether that those splines are in contact with, or spaced-apart from, tissue.

The elements of the electrode assembly are identified from the representation of the element in the ultrasound image and position and orientation data obtained from the ablation catheter. In one embodiment, the medical imaging system employs position and orientation data and electrode assembly shape data from the electrodes, tracking sensors, and other sensors of the ablation catheter and position and orientation data from a tracking sensor on the ICE catheter to determine which elements of the electrode assembly are in view. In another embodiment, the medical imaging system employs image processing of the ultrasound image to determine which elements of the electrode assembly are in view. Mechanisms used to determine whether an electrode or spline is in contact with tissue, such as local impedance data obtained from the electrode assembly, are used to generate the contact visual indicators. Other mechanisms or combinations of mechanisms to determine features of the electrode array are contemplated.

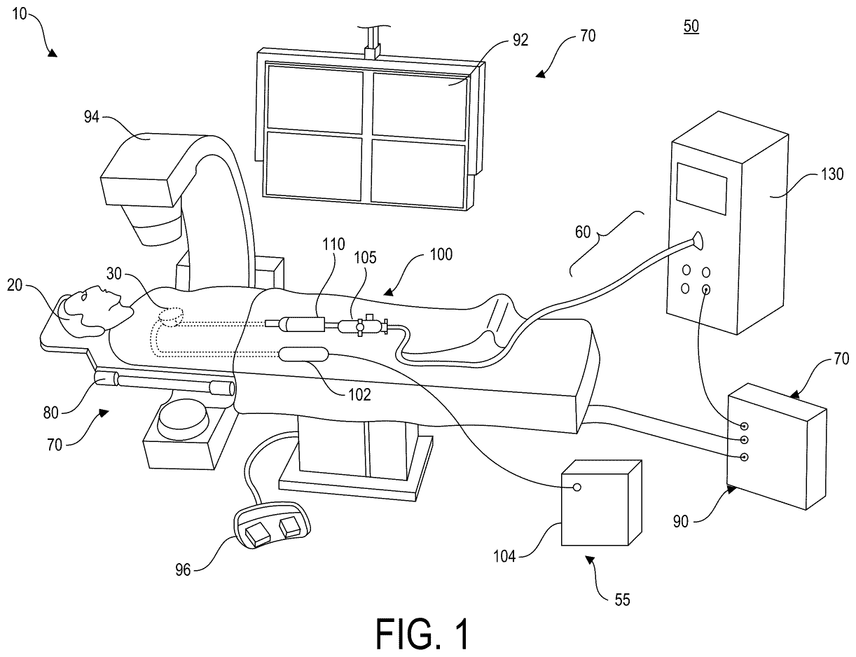

FIG. 1 illustrates an example clinical setting 10 for treating a patient 20, such as for treating a heart 30 of the patient 20, using an electrophysiology system 50, in accordance with the disclosure. The electrophysiology system 50 includes an ablation catheter system, such as an electroporation catheter system 60 in the illustrated embodiment, and an electro-anatomical mapping (EAM) system 70. The example electroporation catheter system 60 includes an electroporation catheter 105, an introducer sheath 110, and an electroporation console 130. (In another embodiment, the ablation catheter system includes a radiofrequency (RF) ablation catheter and an RF signal generator rather than, or in addition to, the electroporation catheter 105 and the electroporation console 130.) Additionally, the electroporation catheter system 60 includes various connecting elements, such as cables and tubing, that operably connect the components of the electroporation catheter system 60 to one another and to the components of the EAM system 70.

Additionally, the clinical setting 10 in the illustrated embodiment includes a medical imaging system 55 configured for use with the electrophysiology system 50. The medical imaging system 55 includes a medical imaging catheter system 102 configured to be inserted into the patient 20 for imaging of portions of the patient's anatomy and a medical imaging console 104 configured to be coupled to the medical imaging catheter system 102 and generate an image on a device, such as display 92. The medical imaging catheter system 102 includes a medical imaging catheter 115, an introducer sheath, and various connecting elements, such as cables and tubing, that connect the components of the medical imaging catheter system 102 to one another and to the components of the medical imaging console 104. In some embodiments, the medical imaging catheter 115 is configured as an ultrasound imaging catheter such as an ICE device or ICE catheter. The medical imaging system 55 can be employed in transseptal catheterization for several percutaneous interventions, including left heart catheter ablation, atrial septal defect closure for effective alternative to surgical intervention. Access to the patient's heart can be obtained through a vessel, such as a peripheral vein often in the groin, such as the femoral vein, or possibly in the shoulder or neck. Once access to the vessel is obtained, the medical imaging catheter 115 can be navigated to within the patient's heart 30, such as within a chamber of the heart.

Also, the clinical setting 10 can include additional equipment such as additional imaging equipment 94 (represented by the C-arm) and various controller elements, such as a foot controller 96, configured to allow an operator to control various aspects of the electrophysiology system 50 and the medical imaging system 55. The clinical setting 10 may have other components and arrangements of components that are not shown in FIG. 1.

The electroporation catheter system 60 is configured to deliver ablation electric field energy to targeted tissue in the patient's heart 30 to create cell death in tissue, for example, rendering the tissue incapable of conducting electrical signals. Also, the electroporation catheter system 60 is configured to generate electric fields using the electroporation catheter 105 to create and present, on the display 92, an electro-anatomical map of the patient's heart to aid a clinician in planning ablation by irreversible electroporation using the electroporation catheter 105 prior to delivering ablation electric field energy. In embodiments, the electroporation catheter system 60 is configured to generate the electric fields based on characteristics of the electroporation catheter 105 and the position of the electroporation catheter 105 in the patient 20, such as in the heart 30 of the patient 20. The electroporation catheter system 60 is configured to generate graphical representations of the electroporation catheter and the electro-anatomical map based on characteristics of the electroporation catheter 105 and the position of the electroporation catheter 105 in the patient 20, such as in the heart 30 of the patient 20, and the characteristics of the tissue surrounding the catheter 105, such as measured impedances of the tissue. The electroporation catheter system 60 can include additional features.

The sheath 110 is operable to provide a delivery conduit through which the electroporation catheter 105 can be deployed to the specific target sites within the patient's heart 30. Access to the patient's heart can be obtained through a vessel, such as a peripheral artery or vein often in the groin or possibly in the shoulder or neck. Once access to the vessel is obtained, the electroporation catheter 105 can be navigated to within the patient's heart, such as within a chamber of the heart.

In one embodiment, the electroporation catheter 105 is a mapping and ablation catheter, which can be deployed in mapping procedures in cooperation with the EAM system 70 as well as to deliver ablation electric field energy and ablate tissue via irreversible electroporation. The example electroporation catheter 105 includes an elongated catheter shaft and distal end region configured to be deployed proximate target tissue, such as within a chamber of the patient's heart, such as the left or right ventricles, or the wall of a pulmonary vein ostium. The shaft can extend from an access point in the patient to the target tissue and generally defines a longitudinal axis of the electroporation catheter 105. A proximal end region of the catheter can include a handle having user manipulatable controls for the catheter 105. The distal end region includes an electrode assembly comprising a plurality of electrodes disposed on splines. For example, the electrode assembly can include a plurality of spaced-apart electrodes or multiple spaced-apart sets or groups of spaced-apart electrodes. In some embodiments, the plurality of spaced-apart electrodes, are deployed on the catheter shaft. The electrode assembly includes a plurality of elements, such as electrodes disposed on a support structure. In the illustrated embodiments, the elements include electrodes and splines, but other elements of the electrode assembly are contemplated, such as alternative support structures such as balloons, or additional elements on the electrode assembly such as flexible circuits, radiopaque markers, echogenic markers, sensors, and other features of the electrode assembly that are identifiable in a visualization such as on an electroanatomical map or ultrasound image.

In one example, the plurality of electrodes can be formed of a conductive, solid-surface, biocompatible material and are spaced-apart across insulators along the shaft of the catheter. Each of the plurality of electrodes is electrically coupled to a corresponding elongated lead conductor that extend along the shaft to a catheter proximal end. In one example, each electrode of the spaced-apart electrodes corresponds with a separate, single lead conductor. In another example, a plurality of electrodes may be coupled to a single lead conductor. Other configurations are contemplated. The plurality of lead conductors can be insulated from one another within an insulating sheath along the catheter shaft, such as with an insulating polymer sheath. The lead conductors can be electrically coupled to plug in the proximal region of the electroporation catheter 105, such as a plug configured to be mechanically and electrically coupled to the electroporation console 130, for example, either directly or via intermediary electrical conductors such as cabling.

The electroporation console 130 includes a controller, such one or more controllers, processors, or computers, that executes instructions or code, such as processor-executable instructions, stored in a non-transitory computer readable medium, such as a memory device, or memory, to cause, such as control or perform, the aspects of the electroporation catheter system 60. In one example, the electroporation console 130 is configured to provide an electrical signal, such as a plurality of concurrent or space-apart-time electrical signals, to the electrically connected electroporation catheter 105 along lead conductors to the spaced-apart electrodes. The spaced-apart electrodes are configured to generate a selected electrical field proximate the target tissue, based on the electrical signals from the electroporation console 130, such as to effect ablation.

The electroporation console 130 can generate electrical signals and select which electrodes in the electrode assembly will receive the electrical signals. A first electrode, or first group of electrodes, can be selected to be an anode and a different, second electrode, or second group of electrodes, can be selected to be a cathode, such that electrical fields can be generated between the anode and cathode based on signals, such as pulses, provided to the electrodes from the electroporation console 130. The console 130 provides electric pulses of different lengths and magnitudes to the electrodes on the catheter 105. The electric pulses can be provided in a continuous stream of pulses or in multiple, separate trains of pulses. Pulse parameters of interest include the number of pulses, the duty cycle of the pulses, the spacing of pulse trains, the voltage or magnitude of the pulses including the peak voltages, and the duration of the voltages. For example, the console 130 can select two or more electrodes of the electrode assembly and provides pulses to the selected electrodes to generate electric fields between the selected electrodes.

In an ablation mode, the console can select electrodes to provide PFA. For example, PFA can be performed with monophasic waveforms and biphasic waveforms. Without being bound to a particular theory, electric field strengths in the range of generally 200-250 volts per centimeter (V/cm) with microsecond-scale pulse duration have been demonstrated to provide reversible electroporation in cardiac tissue. Electric field strengths at approximately 400 V/cm have been demonstrated to provide irreversible electroporation in cardiac tissue of interest, such as targeted myocardium tissue and endocardium tissue, with demonstrable sparing of red blood cells, vascular smooth muscle tissue, endothelium tissue, nerves and other non-targeted proximate tissue.

Additionally, the electrode assembly on catheter 105 can be operated in a selected mode such as monopolar mode or bipolar mode. During monopolar operation of the catheter 105, an electrode, a group of electrodes, or the entire electrode assembly are configured as one of an anode or a cathode. None of the electrodes in the electrode assembly are configured as a the other of the cathode or the anode. Instead, the other of the cathode or the anode is provided in the form of a dispersive pad electrode (not shown) located on the patient, typically on the back, buttocks, or other suitable anatomical location during electroporation. An electrical field is formed between an activated electrode of the electrode assembly and the dispersive pad electrode. In an alternative configuration, a return electrode, such as a plurality of return electrodes, can be disposed on the shaft of the catheter. An electrical field is formed between an activated electrode of the electrode assembly and the return electrode on the shaft of the catheter 105. During bipolar operation of the catheter 105, a first set of one or more electrodes of the electrode assembly, is configured as the anode and a second set of one or more electrodes of the electrode assembly, is configured as the cathode, to generate the electric field. In this example, a pad dispersive electrode is not used, and the electrical field is not extended in the patient's body, but rather through a localized portion of tissue proximate the electrode assembly.

In the illustrated examples, the electroporation catheter 105 is a mapping and ablation catheter, and the electrodes can include ablation electrodes that are configured to deliver ablation electric field energy and mapping electrodes for mapping purposes. In some configurations, the mapping electrodes are configured to be used to collect electrical signals to be used to generate via the operably coupled EAM system 70, and display via the operably coupled display 92, detailed three-dimensional geometric anatomical maps or representations of the cardiac chambers as well as electro-anatomical maps in which cardiac electrical activity of interest is superimposed on the geometric anatomical maps. In some examples, an electrode can operate as an ablation electrode in an ablation mode of the electrophysiology system 50 and as a mapping electrode in a mapping mode of the system 50. Mapping electrodes on the electroporation catheter 105 can measure electrical signals and generate output signals that can be processed by the mapping and navigation controller 90 to generate an electro-anatomical map. In some instances, electro-anatomical maps are generated before ablation for determining the electrical activity of the cardiac tissue within a chamber of interest. In some instances, electro-anatomical maps are generated after ablation in verifying the desired change in electrical activity of the ablated tissue and the chamber. The mapping electrodes may also be used to determine the position of the catheter 105 in three-dimensional space within the body. For example, when the operator moves the distal end of the electroporation catheter 105 within a cardiac chamber of interest, the boundaries of catheter movement can be used by the mapping and navigation controller 90 to form the anatomical map of the chamber. The chamber anatomical map may be used to facilitate navigation of the electroporation catheter 105 without the use of ionizing radiation such as with fluoroscopy, and for tagging locations of ablations as they are completed in order to guide spacing of ablations and aid the clinician in ablating the anatomy of interest. In some examples, an electrode in the electrode assembly can be configured to only perform an ablation or the electrode in the electrode assembly can be configured to only perform mapping.

The EAM system 70 is configured to generate the electro-anatomical map for display on the display 92. The EAM system 70 is operable to track the location of the various components of the electroporation catheter system 60, and to generate high-fidelity three-dimensional anatomical and electro-anatomical maps of the heart, including portions of the heart such as cardiac chambers of interest or other structures of interest such as the sinoatrial node or atrioventricular node. In one illustrative example, the EAM system 70 can include the OPAL™ HDx mapping system marketed by Boston Scientific Corporation. Also, the mapping and navigation controller 90 of the EAM system 70 includes one or more controllers, such as microprocessors or computers, that execute code out of memory to control or perform functional aspects of the EAM system 70, in which the memory, can be part of the one or more controllers, microprocessors, computers, or part of a memory device accessible through a computer network.

The EAM system 70 generates a localization field, via the field generator 80, to define a localization volume about the heart 30, and a location sensor or sensing element on a tracked device, such as sensors on the electroporation catheter 105, generate an output that can be processed by the mapping and navigation controller 90 to track the location of the sensor, and consequently, the corresponding device, within the localization volume. In the illustrated example, the device tracking is accomplished using magnetic tracking techniques, in which the field generator 80 is a magnetic field generator that generates a magnetic field defining the localization volume, and location sensors on the tracked devices are magnetic field sensors.

In other examples, impedance tracking methodologies may be employed to track the locations of the various devices. In such examples, the localization field is an electric field generated, for example, by an external field generator arrangement, such as surface electrodes, by intra-body or intra-cardiac devices, such as an intracardiac catheter, or both. In these examples, the location sensing elements can constitute electrodes on the tracked devices that generate outputs received and processed by the mapping and navigation controller 90 to track the location of the various location sensing electrodes within the localization volume.

The EAM system 70 can be equipped for both magnetic and impedance tracking capabilities. In such examples, impedance tracking accuracy can, in some instances be enhanced by first creating a map of the electric field induced by the electric field generator within the cardiac chamber of interest using a probe equipped with a magnetic location sensor, as is possible using the OPAL HDx™ mapping system. One exemplary probe is the INTELLAMAP ORION™ mapping catheter marketed by Boston Scientific Corporation.

Regardless of the tracking methodology employed, the EAM system 70 utilizes the location information for the various tracked devices, along with cardiac electrical activity acquired by, for example, the electroporation catheter 105 or another catheter or probe equipped with sensing electrodes, to generate, and display via the display 92, detailed three-dimensional geometric anatomical maps or representations of the heart tissue and voids such as cardiac chambers as well as electro-anatomical maps in which cardiac electrical activity of interest is superimposed on the geometric anatomical maps. Furthermore, the EAM system 70 can generate a graphical representation of the various tracked devices within the geometric anatomical map or the electro-anatomical map.

Each cardiac physiological (electrical) signal can include several intracardiac electrograms (EGMs) sensed within a patient's heart and may include any number of features that may be ascertained by aspects of the system 50. Examples of cardiac physiological signal features include activation times, activations, activation waveforms, filtered activation waveforms, minimum voltage values, maximum voltages values, maximum negative time-derivatives of voltages, instantaneous potentials, voltage amplitudes, dominant frequencies, and peak-to-peak voltages. A cardiac physiological signal feature can refer to one or more features extracted from one or more cardiac physiological signals, derived from one or more features that are extracted from one or more cardiac physiological signals. Additionally, a representation, on a cardiac or a surface map, of a cardiac physiological signal feature may represent one or more cardiac physiological signal features, an interpolation of several cardiac physiological signal features. Each cardiac physiological signal also can be associated with a set of respective position coordinates that corresponds to the location at which the cardiac physiological signal was sensed. Each of the respective position coordinates for the sensed cardiac physiological signals can include three-dimensional Cartesian coordinates, polar coordinates, or another coordinate system. The cardiac physiological signals may be sensed on the cardiac surfaces, and the respective position coordinates can be on the endocardial surface, epicardial surface, in the mid-myocardium of the patient's heart, or in a vicinity.

During a signal-acquisition stage of a cardiac mapping procedure, the electroporation catheter 105 is displaced to multiple locations within the heart chamber into which the catheter 105 is inserted. At each location to which the catheter 105 is moved, the electrodes and sensors acquire physiological signals resulting from the electrical activity in the heart along with positional, or spatial, information of the catheter 105. The spatial information is used in building a three-dimensional grid of the anatomy during mapping. To perform a mapping procedure and reconstruct physiological information on the endocardium surface, the EAM system 70 may align a coordinate system of the catheter 105 with the endocardium surface's coordinate system, or vice versa. Alternatively, or additionally, the grid may be used to capture EGMs, and select mapping values based on statistical distributions associated with nodes of the grid. The EAM system 70 also can perform post-processing operations on the physiological information to extract and display useful features of the information to the operator of the system 50.

In generating an example electroanatomical map, a data stream including multiple signals, such as signals received from the mapping electrodes of the catheter 105, is input into the EAM system 70. During the automated electroanatomical mapping process, the data stream provides a collection of physiological and location signals that serve as an input to the mapping process. The signals may be collected directly by the mapping system, obtained from another system using an analog or digital interface, or both. The data stream can include signals such as unipolar and/or bipolar intracardiac EGMs, surface electrocardiograms (ECGs), electrode location information originating from one or more of a variety of methodologies, tissue proximity information, catheter force information, catheter to tissue contact information, catheter temperature, acoustic information, catheter electrical coupling information, catheter deployment shape information, electrode properties, respiration phase, blood pressure, and other physiological information. For the generation of specific types of maps, one or more signals may be used as one or more references to trigger and align the data stream relative to a cycle or clock, which can be used to create beat datasets. Beat metrics can be determined from the beat datasets. A beat acceptance process can be applied to determine which beat datasets will make up a map dataset. The map dataset may be stored in association with a three-dimensional grid that is dynamically generated during data acquisition.

Surface geometry data of the cardiac surface is generated, such as generated concurrently, during the data acquisition process using acceptance metrics employing a surface geometry construction process. This process constructs surface geometry using data such as electrode locations and catheter shape contained in the data stream. Additionally, or alternatively, previously collected surface geometry of the cardiac surface can be used as an input to surface geometry data. Previously collected geometry may have been collected using a different map dataset or using a different modality such as computerized tomography (CT), magnetic resonance imaging (MRI), ultrasound, or rotational angiography and registered to the catheter locating system. A surface map generation process is employed to generate surface map data from the map dataset and surface geometry data.

The depiction of the electrophysiology system 50 is intended for illustration or a general overview of the various components of the system 50 and is not intended to imply that the disclosure is limited to any set of components or arrangement of the components. For example, additional hardware components, such as breakout boxes or workstations, can be included in the electrophysiology system 50.

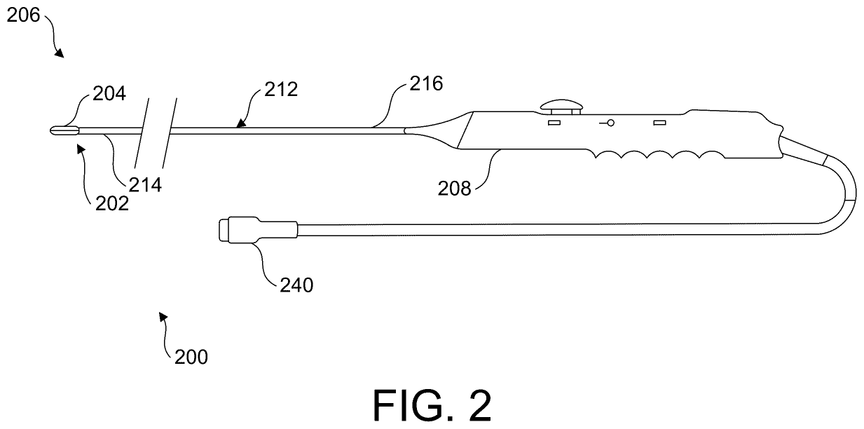

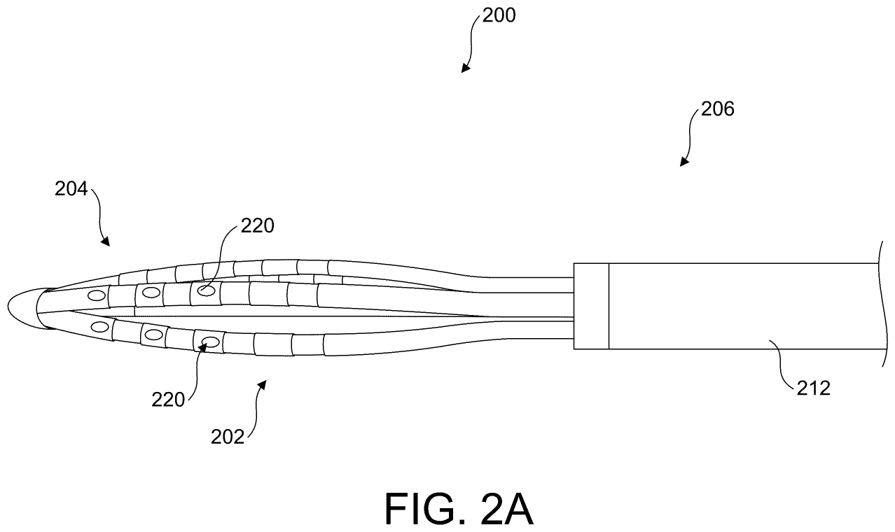

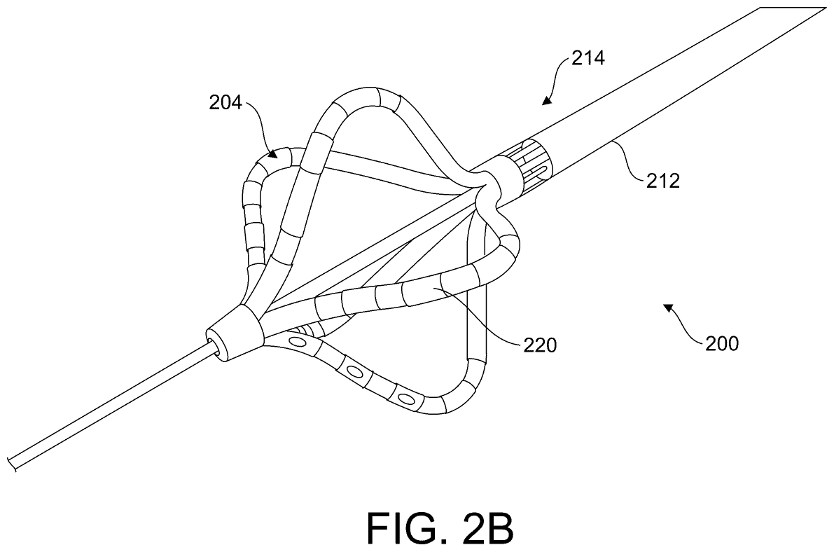

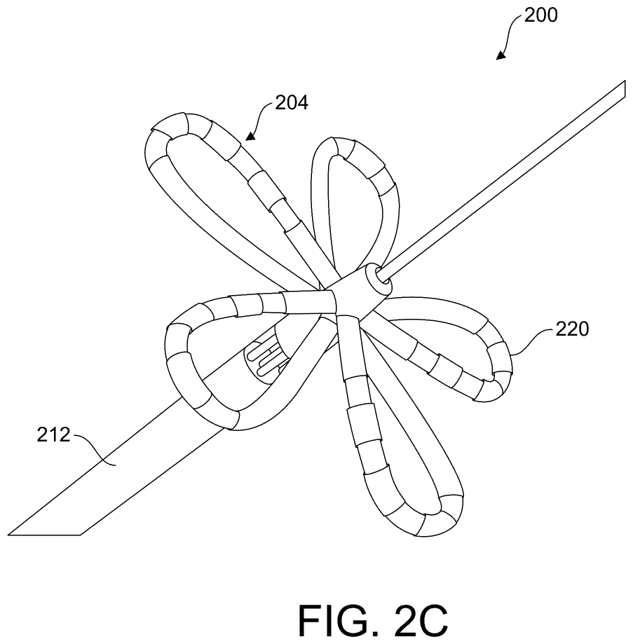

FIG. 2 is a side view of an embodiment of a mapping and ablation electroporation catheter 200, which corresponds with electroporation catheter 105 of FIG. 1. The electroporation catheter 200 includes an electrode assembly 202 of an example of a dynamic catheter, dynamic electrode assembly, or expandable electrode assembly. A dynamic catheter, dynamic electrode assembly, or expandable electrode assembly, as used in this disclosure, includes an electrode assembly that can be made to assume a plurality of configurations such as in a collapsed configuration and an expanded configuration in which the electrodes are movable away from and back toward the longitudinal axis. The dynamic catheter is distinguishable from a static catheter, or rigid catheter, in which the electrode array that comprises only linearly spaced-apart electrodes along a distal portion of the catheter shaft. The electroporation catheter 200 can assume a plurality of configurations including a collapsed configuration and multiple expanded configurations, such as an intermediate configuration and fully expanded configuration. FIG. 2 illustrates the catheter 200 in the collapsed configuration. The catheter 200 includes an electrode assembly 202 comprising a set of splines 204 at a distal end 206 of the catheter 200 and a handle 208 at a proximal end 210 of the catheter 200. Each spline of the set of splines 204 includes one or more electrodes formed on a surface of the spline. The electrodes can be configured as ablation electrodes for performing an ablation, sensing electrodes for performing mapping or detection of physiological signals, either ablation or sensing electrodes, or a combination of ablation electrodes and sensing electrodes. Each spline of the set of splines 204 can be configured to include a flexible curvature so as to rotate, or twist and bend and form a petal or tear drop-shaped curve in a fully expanded configuration. The catheter 200 includes a catheter shaft 212 defining a longitudinal axis. The shaft 212 includes a distal region 214 and a proximal region 216. A shaft lumen extends longitudinally through the shaft 212 from the distal region 214 to the proximal region 216 along the longitudinal axis. In some embodiments, a diameter of the catheter shaft 212 is between about 6 Fr. and about 15 Fr. In some embodiments, the catheter shaft 212 has a length of between about 85 cm and about 135 cm.

The set of splines 204 extend from the distal region 214 of the shaft 212. In exemplary embodiments, the set of splines 204 includes a proximal end affixed to the distal region 214 of the shaft 212. The splines include distal ends tethered or fixed to a distal cap such that the set of splines 204 and distal cap can translate relative to the catheter shaft 212 so as to expand and contract together. In various embodiments, the distal cap translates longitudinally along the axis with respect to the shaft. The proximal ends of the set of splines 204 are configured for translation along the longitudinal axis to transition the electrode assembly 202 between the collapsed configuration and the expanded configuration. In some examples, the set of splines are configurable in an intermediate configuration for use such as ablation and mapping. The intermediate configuration is between the collapsed configuration and the expanded configuration, and the set of splines can be arranged in, for example, a basket or spherical configuration.

The proximal region 216 of the catheter shaft 212 in some embodiments is coupled to a handle 208 at the proximal end 210 of the catheter 200. The handle 208 is operably coupled to the set of splines 204 and the distal cap. For example, the handle 208 includes a translation member disposed in the handle 208. The translation member can include a knob, such as a thumb-activated slider, configured to be manipulated translationally or rotationally by a clinician. In the illustrated embodiment, the translation member is configured for translation along the longitudinal axis by moving or sliding the knob between a plurality of positions to transition the electrode array 202 between a set of configurations. The handle 208 can include a cable having connector 240. The connector 240 are configured to couple to an extension cable to connect the catheter 200 to a console, such as console 130, or other components. In some embodiments, connector 240 is electrically connected to electrical components such as the electrodes, impedance tracking sensors, a magnetic sensor, temperature sensor, a gyroscopic sensor, an accelerometer, an electrode or other device to detect the shape of the splines via leads disposed in the handle 208, shaft 212, or on the electrode assembly 202.

FIGS. 2A, 2B, and 2C illustrate the electrode assembly 202 at the distal end 206 of the catheter 200 corresponding with FIG. 2. More particularly, FIG. 2A illustrates the electrode assembly 202 in a collapsed configuration, FIG. 2B illustrates the electrode assembly 202 in the intermediate, or basket, expanded configuration, and FIG. 2C illustrates the electrode assembly 202 in a fully expanded configuration. In the illustrated embodiments, a translatable shaft extends from catheter shaft 212, such as from within a lumen of the catheter shaft 212 and is movable with respect to the catheter shaft 212. In embodiments, the translatable shaft is included with the translation member and mechanically coupled to the knob. As the knob is moved from a collapsed configuration position to the intermediate configuration position to the expanded configuration position on the handle 208, the translatable shaft is pulled within the lumen of the catheter shaft 212 or retracted into the catheter shaft 212. As the knob is moved from the expanded configuration position to the intermediate configuration position to the collapsed configuration position, the translatable shaft is pushed out of the lumen of the catheter shaft 212 or extended out of the catheter shaft 212. FIG. 2A illustrates the set of splines 204 of the electrode array 202 in the collapsed configuration in which the set of splines 204 define a longitudinally extending cylinder such that the set of splines 204 can be advanced through a patient's vasculature. The spline 204 carry electrodes 220, which can be configured as ablation electrodes in the illustrated embodiment. FIG. 2B illustrates the set of splines 204 of the electrode array 202 in the intermediate configuration in which the set of splines 204 define a basket. The splines 204 are curved and are operational to perform a mapping or ablation, or a therapeutic, mapping or diagnostic function. FIG. 2C illustrates the set of splines 204 of the electrode array 202 in the expanded configuration in which the set of splines 204 define a flower. The curves of the splines 260 are most pronounced in the expanded configuration. The set of splines 204 are operational to perform a mapping or ablation.

The embodiment of the ablation catheter, or mapping and ablation catheter, is presented for illustration only. Embodiments of the catheter for use with this disclosure include therapeutic, mapping or diagnostic catheters. For instance, embodiments of catheters for use with this disclosure include cardiac catheters. Additionally, location tracking via magnetic sensors is presented for illustration only. Embodiments of the catheter for use with this disclosure can include various location tracking mechanisms including magnetic sensors, EAM electrodes, and other tracking technologies now known or later discovered.

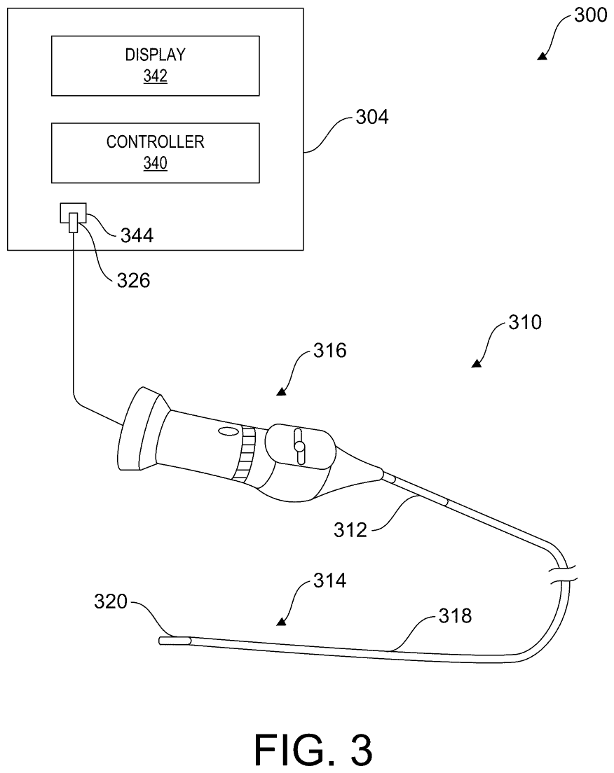

FIG. 3 illustrates an embodiment of a medical imaging system 300, including a medical imaging console 304 and medical imaging catheter 310 corresponding with the medical imaging console 104 and medical imaging catheter 115 medical imaging system 55 of FIG. 1. The medical imaging system 300 is suitable for use in a clinical setting for a medical procedure on a patient, such as a medical procedure, including an electrophysiology procedure or intracardiac procedure, on a heart of the patient for generating a visualization such as an ultrasound image. The medical imaging catheter 310 includes a proximal end region 312 and a distal end region 314. The medical imaging catheter 310 defines a longitudinal axis. The proximal end region 312 of the medical imaging catheter 310 includes a catheter hub 316, and the distal end region 314 is configured and arranged to be inserted into the patient. The medical imaging catheter 310 includes a shaft 318 that is dimensioned to be inserted within regions of the patient that are difficult to navigate, including blood vessels, heart chambers, and, in some cases, the gastrointestinal tract, the urinary tract, and the pulmonary system.

The distal end region 314 includes an imaging module 320 that can be, for example, extended into the patient's heart and disposed at the end of the shaft 318. In the illustrated embodiment, the imaging module 320 includes an imaging module cover forming a distalmost tip of the medical imaging catheter 310. The imaging module includes an imaging transducer configured for imaging the patient's anatomy such as target anatomy proximate the distal end region 314. In embodiments, the imaging module 320 includes an ultrasound imaging device configured to transmit and receive acoustic energy to generate ultrasound images of the target anatomy. The imaging transducer is coupled to elongated lead conductors that extend from the distal end region 314 along the catheter shaft 318 to the proximal end region 312, such as to a plug 326 at the proximal end region 312. The distal end region 314 in embodiments also includes a magnetic field tracking sensor coupled to elongate lead conductors such that the medical imaging catheter 310 is equipped with magnetic tracking capability to detect position and orientation of the distal end region, such as the imaging transducer, with respect to the magnetic field defining the localization volume like the electroporation catheter 200. In other examples, the medical imaging catheter is equipped with impedance tracking capability. In one embodiment, the lead conductors include electrically conductive elements, configured to carry an electrical signal, such as wires or traces that are electrically insulated from one another within an insulative sheath, such as with an insulative polymer sheath, or disposed on a flex circuit extending the length of the shaft 318. The plug 326 is configured to be mechanically and electrically coupled to the medical imaging console 304, for example, either directly or via intermediary cabling.

In the illustrated embodiment, the hub 316 includes a housing forming handle assembly and a steering control unit with a steering actuator. The steering control unit is provided for articulating the distal end region 314 and positioning the imaging module 320. The steering control unit includes, in some embodiments, steering cables coupled to the steering actuator and disposed within the shaft 318 extending to the distal end region 314. The steering actuator can be rotated to facilitate positioning of the imaging module 320. When the clinician rotates the steering actuator, a steering cable pulls the distal end region to bend the shaft 318 to select a position of the imaging module with respect to the target anatomy.

The medical imaging console 304 includes an imaging controller 340 coupled to a imaging catheter control circuit 342. The medical imaging console 304, includes a receptacle 344 configured to couple to cabling or plug 326 and a display 342, or is configured to be coupled to a display 342 via a display connection. The imaging controller 340 is implemented with any combination of hardware and programming to receive inputs from and provide outputs to catheter control circuit, and to provide outputs to and, in some embodiments, receive inputs from a display. The imaging controller 340, such one or more controllers, processors, or computers, executes instructions or code, such as processor-executable instructions, stored on a non-transitory computer readable medium, such as a memory device, or memory, to cause, such as control or perform, the aspects of the medical imaging catheter system 300. The imaging controller 340 includes input and output ports that allows the imaging controller 340 to control or receive inputs from the imaging catheter control circuit and generate a visualization for facilitation on the display according to a programmed scheme. The controller 340 is configured to provide a signal, such as a plurality of concurrent or space-apart-time electrical signals, to the connected medical imaging catheter 310 via the receptacle 342 that is transmitted along the lead conductors to the imaging module 320. The imaging catheter control circuit is interposed between the imaging controller 340 and the catheter 110 and includes a transmit beamformer and a receive beamformer. The transmit beamformer is configured for transmission of an electrical signal or electrical impulse towards the ultrasound imaging device 320 of the medical imaging catheter 310. The receive beamformer 346 is configured to receive an electrical signal or electrical impulse from the ultrasound imaging device 320.

In one embodiment, the ultrasound imaging device is disposed within the imaging module 320 with respect to the longitudinal axis in such a manner to produce a side image, such as an image perpendicular to the longitudinal axis. The ultrasound imaging device includes an operable or active surface configured to produce an acoustic signal and receive echoes that are aligned parallel with the longitudinal axis. The ultrasound imaging device, in some embodiments, provides a wedge-shaped image, such as a ninety-degree sector plane, from the side of the distal end of the medical imaging catheter 310 that can be displayed on the medical imaging console 304. In one example of the ultrasound imaging device, a two-dimensional imaging phased array device, which include one-dimensional arrays, creates two-dimensional images by steering an acoustic beam across a two-dimensional plane. Each acoustic beam, or scan line, produces echoes that are measured and combined with other scan lines into an ultrasound image. To create a three-dimensional or four-dimensional (including time as a dimension) image, a two-dimensional array is applied to steer the beam throughout a three-dimensional volume or two orthogonally disposed one-dimensional arrays scan each scan beams across a respective two-dimensional plane. The ultrasound imaging device still produces a wedge-shaped image, such as a ninety-degree sector plane, which is plus/minus forty-five degrees in the axis perpendicular to the longitudinal axis.

The imaging controller 340 is configured to generate an ultrasound image for facilitation on the display. In embodiments, the ultrasound image is based on a mode of the medical imaging system 300. In one embodiment, the imaging controller 340 is configured to operate in cooperation with the ultrasound imaging device in at least one of a plurality of modes, such as an amplitude mode (A-mode), a motion mode (or M-mode), a brightness mode (or B-mode), and a Doppler mode including pulse wave Doppler, continuous wave Doppler, spectral Doppler, and color Doppler. In one example configuration, the visualization is presented in B-mode.

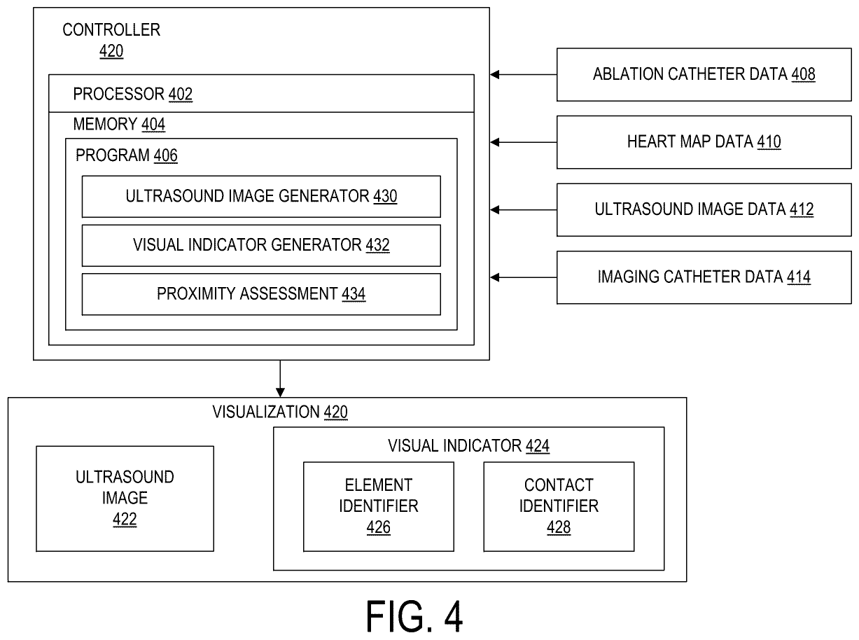

FIG. 4 illustrates an example medical imaging controller 400 that can be used with the example electrophysiology system 50. In some embodiments, the controller 400 is implemented in a single device, such as the mapping and navigation controller 90 of the EAM 70, the medical imaging console 304 of the medical imaging system 300, the electroporation console 130, or some other controller. In embodiments, the controller 400 is a distributed controller, such as a controller implemented in the mapping and navigation controller 90 and the medical imaging console 304. The controller 300 can be implemented to include a visual indicator, such as an overlay, to an ultrasound image for facilitation on a display. Further, the controller 400 is implemented to provide a visual indicator of which of the ablation electrodes of an electroporation catheter, such as catheter 200, is in contact with target tissue. The controller 400 can include a processor 402 and a memory 404. The memory 404 stores processor executable instructions 406. In one example, the processor executable instructions 406 can be in the form of a program, such as a computer program or application. The processor 402 can execute the instructions 406 that can be included in configuring the controller 400. In one example, the controller 400 can be implemented to include a computing device such as a laptop computer, a workstation, a desktop computer, a tablet, or a smartphone or devices such the mapping and navigation controller 90 and the medical imaging console 304. In embodiments, the controller 400 can include additional components such as a display, a touchscreen, speakers or other output devices, a keyboard or other input devices, or communication circuitry such as computer network adapters. The controller 400 may be implemented in a variety of architectures and components, such as the processor 402, memory 404, and program 406 may be distributed in various locations.

In one example, the processor 402 may include a plurality of main processing cores to run an operating system and perform general-purpose tasks on an integrated circuit. The processor 402 may also include built-in logic or a programmable functional unit, also on the same integrated circuit. In additional to multiple general-purpose, main processing cores and the application processing unit, controller 400 can include other devices or circuits such as graphics processing units or neural network processing units with the main processing cores. For example, the controller 400 may be used to perform other tasks.

Memory 404 is an example of computer storage media. Computer storage media includes RAM, ROM, EEPROM, flash memory or other memory technology, CD-ROM, digital versatile discs (DVD) or other optical storage, magnetic cassettes, magnetic tape, magnetic disk storage or other magnetic storage devices, USB flash drive, flash memory card, or other flash storage devices, or other storage medium that can be used to store the desired information and that can be accessed by the processor 402. Any such computer storage media may be part of the controller 400 and implemented as memory 404. Memory 404 is a non-transitory, processor readable memory device. Accordingly, a propagating signal by itself does not qualify as storage media or memory 404.

The controller 400 is configured to receive inputs or information from the electrophysiology system 50, such as inputs from the electroporation catheter system 60 and EAM system 70 including the electroporation console 130 and the mapping and navigation controller 90 and the medical imaging console 304, for storage in memory 404 and use by the instructions 406. In some embodiments, the controller 400 receives inputs representative of data obtained with the catheter system 60, such as data determined from electrical or physiological signals received from electrodes on the catheter assembly such as the ablation electrodes and sensing electrodes, and tracking devices, and other data, or ablation catheter data 408. Ablation catheter data 408 can include data determined from physiological signals of the heart, electrode location information, tissue proximity information, force or contact information, catheter tip or tissue temperature, acoustic information, catheter electrical coupling information, catheter deployment shape information, electrode properties, respiration phase, blood pressure, impedance information and other physiological information. In some embodiments, electroporation catheter obtained data 408 includes or is supplemented with other information collected from sensors during mapping of the heart. In some embodiments, the controller 400 receives an input representative of the anatomical map of the heart, or heart map data 410, which collected heart map data 410 can include the data regarding representations of the geometric anatomical map of the heart and the electro-anatomical map of the heart, such as from the EAM system 70. Heart map data 410 can include data previously collected, such as in the same procedure using a different map dataset, or with a different modality such as computerized tomography (CT), magnetic resonance imaging (MRI), ultrasound, or rotational angiography, and registered to the catheter locating system. Additionally, heart map data 410 can include annotations, markings, or user-added tags of the associated with the anatomical map of the heart that may include markings of anatomical locations of interest or other data to generate visualizations a clinician may find of interest during a procedure. Further, the controller 400 receives inputs representative of ultrasound images obtained with the medical imaging system 300 as an ultrasound image data 412. In embodiments, the controller 400 includes signals to generate an ultrasound image or facilitate for display an ultrasound image. Still further, the controller receives inputs representative of position and orientation of the medical imaging catheter, such as data from the tracking sensors, such as imaging catheter data 414.

The controller 400 is configured to generate outputs such as a visualization 420. The controller is configured to generate a visualization 420 including an ultrasound image 422, such as image of the anatomy in B-mode. The controller 400 is also configured to generate a visual indicator 424 that provides information regarding the electrode assembly of the ablation catheter in the ultrasound image as an overlay of the ultrasound image 422. In one embodiment, the visual indicator 424 includes an electrode assembly element identifier 426. The electrode assembly element identifier 426 identifies elements of the electrode assembly, such as splines or electrodes, that are in view on the ultrasound image 422. In one embodiment, the visual indicator 424 includes a contact visual identifier 428. The contact visual identifier 428 indicates whether a spline or electrode in the ultrasound image 422 is within a predetermined proximity to tissue, such as in contact with tissue.

The program 406 can include a suite of modules stored in memory 404. For instance, the controller 400, via the program 406, can be configured to generate an ultrasound image from signals received from the ultrasound device with an ultrasound image generator 430. The program 406 can be configured to generate a visual indicator with an visual indicator generator 432. For instance, the visual indicator generator 432 can receive the ultrasound image from the ultrasound image generator.

In some embodiments, the program 406 is configured to determine whether an electrode is within a predetermined proximity to tissue, such as in contact with tissue, with a proximity assessment engine 434. The proximity assessment engine 434 can be included in program 406 or implemented with the EAM system 70 or electroporation system 60, and an assessment of proximity provided to program 406. For example, the EAM system 70 can determine whether electrodes are within the predetermined proximity to tissue, and then provide data as an input to program 406 as to which electrodes are within the predetermined proximity for use with the contact visual identifier 428 as proximity assessment data 416. proximate electrical signals with a pre-pulser to provide to the catheter 105 and receive feedback from anatomy as part of catheter obtained data 408. The pre-pulser, via the electroporation console 130, can provide electrical signals to the ablation electrodes only in some embodiments and configured in a bipolar mode to determine impedance or current readings from feedback signals. In some embodiments, the pre-pulser, via the electroporation console 130, provides electrical signals to the ablation electrodes and the sensing electrodes configured in a bipolar mode to determine impedance or current readings from feedback signals. The pre-pulse electrical signal can be a continuous signal that is selectively read or can be a selectively applied, discrete pre-pulse signal that is read in response to being applied. The program 406 includes a calibrator to obtain a baseline measurement, such as readings when the catheter is not in contact with tissue that can be compared to readings when the catheter is in contact with tissue to determine which of the ablation electrodes are in contact with tissue. In one example, the calibrator is operative with the pre-pulser to provide, for instance, a first pre-pulse signal with the catheter not in contact with the target tissue and a second pre-pulse signal with the catheter in contact with the target tissue, such as with a selectively applied, discrete pre-pulse signal. Additionally, the program 406 can include one or more tissue contact assessment processes that can be applied to determine which of the ablation electrodes are in contact with the target tissue. In the case of a plurality of tissue contact assessment processes, and the controller 400 is further configured to select a tissue contact assessment process from the plurality of tissue contact assessment processes. In some embodiments, the program is included in as part of an EAM package.

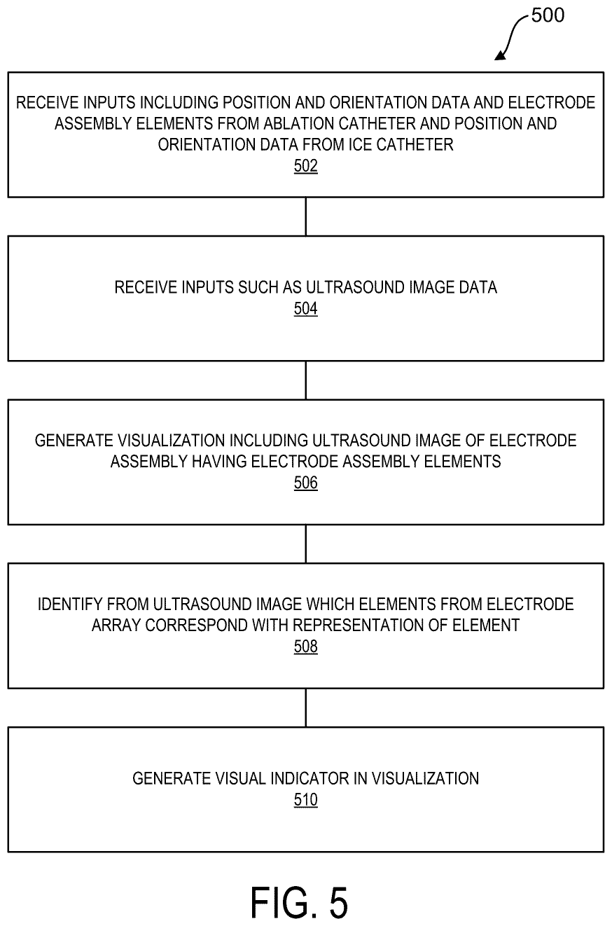

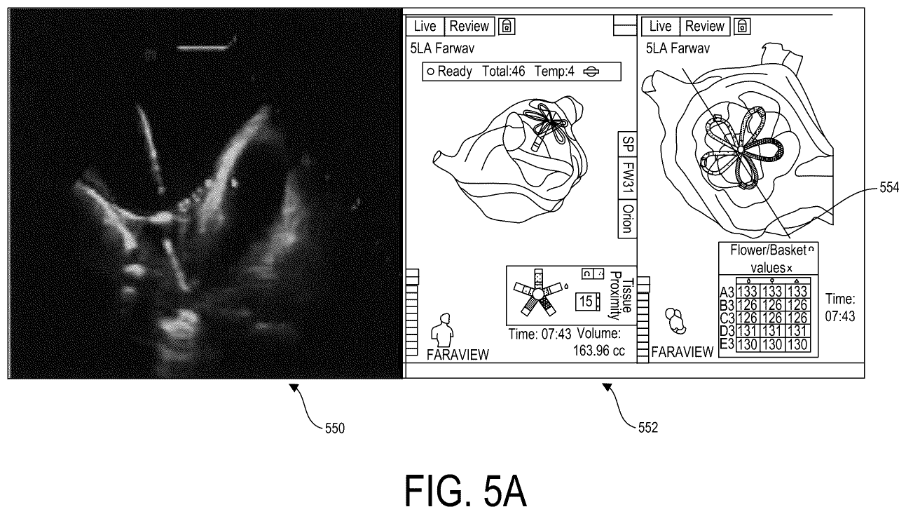

FIG. 5 illustrates a process 500 of configuring the controller 400 for use in generating a visualization including an ultrasound image and visual indicators identifying elements of an electrode assembly of an ablation catheter used in a surgical procedure. In embodiments, the process 500 is a computerized process. In one embodiment, process 500 is implemented as set of processor-executable instructions, such as instructions 406, stored in a non-transitory memory, such as memory 404 to be executed by a processor 402 to configure controller 400. The instructions 406 to implement process 400 can be configured to receive data, such as to retrieve from memory 404 ablation catheter data 408, heart map data 410, ultrasound image data 412, and imaging catheter data 414. In some embodiments, the program 406 retrieves from memory 404 proximity assessment data 416. Further, the instructions 406 to implement process 400 can be configured to generate a visualization, such as visualization 420 on a display of graphical representations on display device.

The controller receives data regarding the position, orientation and configuration of the dynamic elements of the electrode assembly and position and orientation information of the ultrasound imaging catheter at 502. For example, controller receives ablation catheter data 408 including electrode location information, tissue proximity information, force or contact information, catheter deployment shape information (electrode assembly shape information), such as from tracking sensors, force detection sensors, electrode assembly shape detection configurations, and other information. The controller also receives imaging catheter data 414. In addition, the controller 400 receives an input representative of the anatomical map of the heart, or heart map data 410, which collected heart map data 410 can include the data regarding representations of the geometric anatomical map of the heart and the electro-anatomical map of the heart, such as from the EAM system 70 as well as inputs representative of ultrasound images obtained with the medical imaging system 300 as an ultrasound image data 412 as input data at 504.

A visualization is generated in which the visualization includes an ultrasound image of the electrode assembly having a plurality of elements at 506. In some embodiments, the ultrasound image includes a two-dimensional imaging plane having a representation of an element of the electrode assembly, such as a representation of an element of the electrode assembly with respect to tissue. In some examples, more than one of the elements are in the ultrasound image. The elements correspond with an element of the electrode assembly the controller is configured to identify. For instance, the controller could be configured to identify a structural feature of the electrode assembly such as a spline, an electrode, a marker, such as an echogenic element disposed on the electrode assembly.

An identification from the ultrasound image is made as to which element of the electrode assembly corresponds with the representation of the element of the electrode assembly and position and orientation data of the ablation catheter at 508. The identification of the element or elements on the ultrasound image can be achieved via one or more of several mechanisms. In one embodiment, the identification implements shape detection information regarding the electrode assembly and position and orientation information regarding the electrode assembly and the imaging catheter. In one implementation, shaped detection information is used to determine the configuration of the electrode assembly, such as whether the electrode assembly is in the collapsed configuration or in one of the at least one expanded configurations. In one implementation, shape detection information includes impedance measurements between ablation or sensing electrodes on the electrode assembly and a reference electrode to determine the shape of the elements, such as the splines, on electrode assembly. In some embodiments, additional image processing, such as segmentation techniques, are applied. In one application, the elements of the electrode assembly are identified from the ultrasound image via image processing techniques. For examples, the program can apply rule-based, deterministic, or condition processing in the methods to identify the elements of the electrode assembly in the ultrasound image. In another example, the program can apply artificial intelligence such as machine learning in the method to identify the elements. In some applications, the identification is based on the three-dimensional position of the ablation catheter with respect to tissue as provided in the anatomical map of the EAM system 70. In another application, the identification is based on a determination of a subset of intersecting points between the imaging catheter and the ablation catheter based on the three-dimensional anatomical map, or EAM space. Transformations, or inverse transformations, are applied to project the points into the two-dimensional ultrasound image. In some embodiments, post process error corrections are applied based on object detection or image segmentation tracking processing. In one embodiment, an identifier is assigned to each element of the plurality of elements of the electrode assembly, such as determined from the electroanatomical map. A determination is made, from the ultrasound image, as to which identifier corresponds with the representation of the element of the electrode assembly.

In some embodiments, identification of the element of the electrode assembly includes a determination as to whether the element is within a predetermined proximity to tissue, such as whether an ablation electrode is in contact with tissue. In one embodiment, the predetermined proximity is based on whether the position of the ablation electrode with respect to the tissue is close enough to provide an effective lesion resulting from determined pulse parameters. In one embodiment, contact with the tissue is within a predetermined proximity. An activated ablation electrode in contact with tissue will provide an effective lesion. In another example, an activated ablation electrode in a blood pool and not within the predetermined proximity to tissue can cause hemolysis, which can have adverse effects for a patient including acute kidney injury. In one embodiment, the predetermined proximity is a distance. In another embodiment, the predetermined proximity is based on an impedance measurement. For instance, an impedance measurement is indicative of proximity to tissue, and a determination of whether the impedance is indicative of a predetermined proximity is made based on whether the impedance is within a range of impedances. In some embodiments, the controller is also configured to determine which of the ablation electrodes, if any, are in a second subset of ablation electrodes are not within the predetermined proximity to the planned ablation zone. Exemplary methodologies for identifying the tissue-contacting ablation electrodes are known.

FIG. 5A includes a schematic drawing of an ultrasound image 550 of an ablation catheter having a dynamic electrode assembly in a cardiac chamber and a visualization of an electroanatomical map 552 of the cardiac chamber with the ablation catheter to illustrate an identification at 508. The two-dimensional ultrasound image 550 represents a wedge-shaped slice of three-dimensional space, which is represented in the EAM visualization 552. By way of example, the two-dimensional ultrasound imaging plane 554 is indicated on the EAM visualization 552. The position and orientation of the ablation catheter within the three-dimensional space is determined from ablation catheter data 408, such as magnetic tracking information and shape detection information. Additionally, the position and orientation of the imaging catheter within the three-dimensional space is determined via imaging catheter data 414, such as magnetic tracking information. Magnetic tracking information, for example, can encode six degrees of freedom including three for translation and three for rotation. The received data at 502, 504 is applied to determine the spatial correspondence between the ultrasound image information and the EAM localization frame. In one embodiment, each pixel in the ultrasound image data is associated with a location in the three-dimensional space of an EAM coordinate system. Tracking the location of an image pixel in the EAM coordinate, in one embodiment, includes a chain of concatenated transformations and error corrections as is known in the art.

Referring to FIGS. 5 and 5A, a visual indicator is generated in the visualization in which the visual indicator associated with the identified element of the electrode assembly at 510. In one embodiment, the visual indicator includes an electrode assembly element identifier that identifies features of the electrode assembly, such as splines or electrodes, in view on the ultrasound image. In one embodiment, the visual indicator includes a contact visual identifier that indicates whether a spline or electrode in the ultrasound image is in contact with tissue. In an embodiment, the visualization includes a corresponding visual indicator in a representation of the element in a graphical representation generated via the EAM system 70. For example, the EAM visualization 552 includes representations of the electrodes or splines in the ultrasound imaging plane 554, such as intersectional representations of the electrodes or splines in the ultrasound imaging plane 554. In the illustrated EAM visualization 552, two splines are presented in the ultrasound imaging plane 554. The splines or electrodes in the EAM visualization 552 are identified via a particular color or other indicia. In one example, one spline in the ultrasound imaging plane 554 is colored red and the other spline in the ultrasound imaging plane 554 is colored blue. The ultrasound representation of the splines in the ultrasound image 550 can include the same or substantially the same indicia, such as the spline in the ultrasound image 550 corresponding with the red colored spline in the EAM visualization 552 is also colored red and the spline in the ultrasound image 550 corresponding with the blue colored spline in the EAM visualization 552 is also colored blue. In this embodiment, the ultrasound imaging catheter has an orientation with respect to the cardiac anatomy and is configured to include image the electrophysiology catheter. One or more elements of the electrophysiology catheter, such as elements of the electrode assembly, in the ultrasound image 550 are each represented with an associated indica. The current, or real time, two-dimensional ultrasound imaging plane 554 in the EAM visualization 552 is associated with the current, or real time, orientation of the ultrasound imaging catheter. A first representation of a catheter element, such as a first element of the electrode assembly that intersects with the ultrasound imaging plane 554 in the EAM visualization 552 is represented with an indicia similar or the same as in the ultrasound image 550. A second representation of a catheter element, such as a second element of the electrode assembly that intersects with the ultrasound imaging plane 554 in the EAM visualization 552 is represented with an indicia similar or the same as in the ultrasound image 550.

In one embodiment, the visual indicator is presented as an overlay on the ultrasound image of the electrode assembly generated in the visualization at 506. In some embodiments, the visual indicator is direct overlay such as the representation of the element in the ultrasound image is highlighted, defined, or colored so as to appear highlighted, defined or colored on the ultrasound image. Examples of a direct overlay include the visual indicators highlight approximate areas of the identified elements, delineate individual splines, and delineate individual electrodes. In another embodiment, the visual indicator is an indirect overlay, such as an indirect indication not presented on the representation of the elements in the ultrasound image. Examples include a text message such as “BLUE SPLINE IN PLANE” or an inset with the visual indicators highlighting spline location.

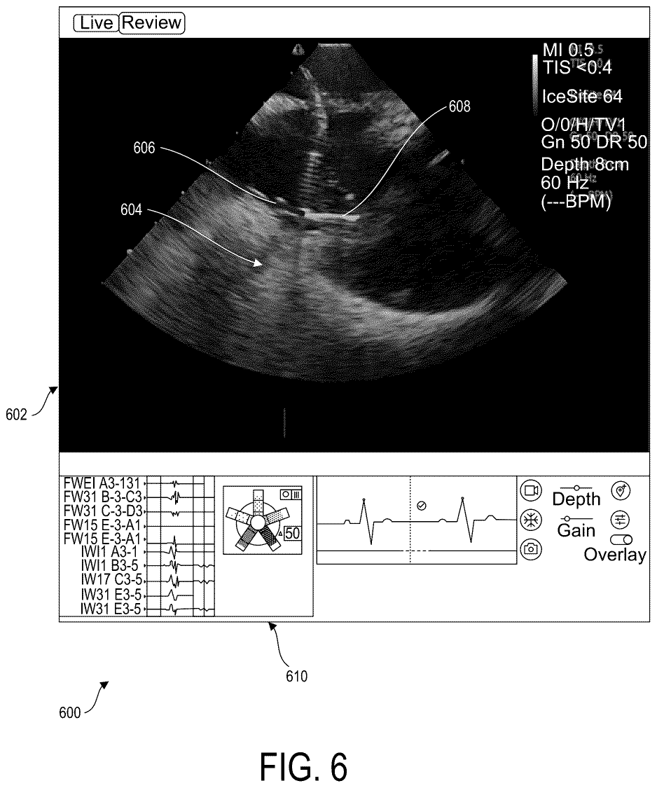

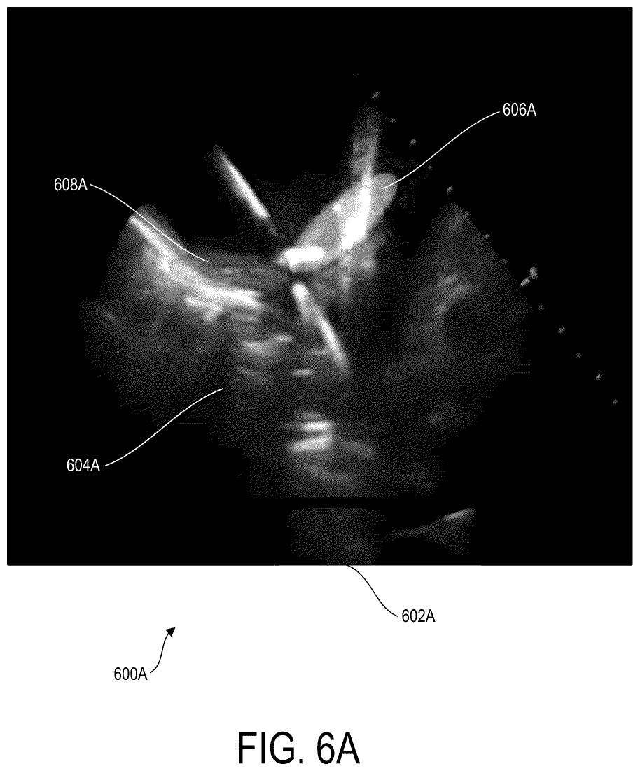

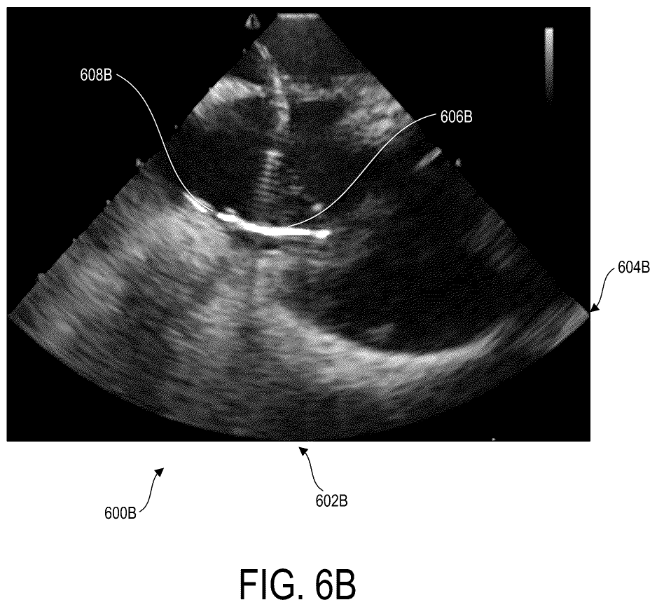

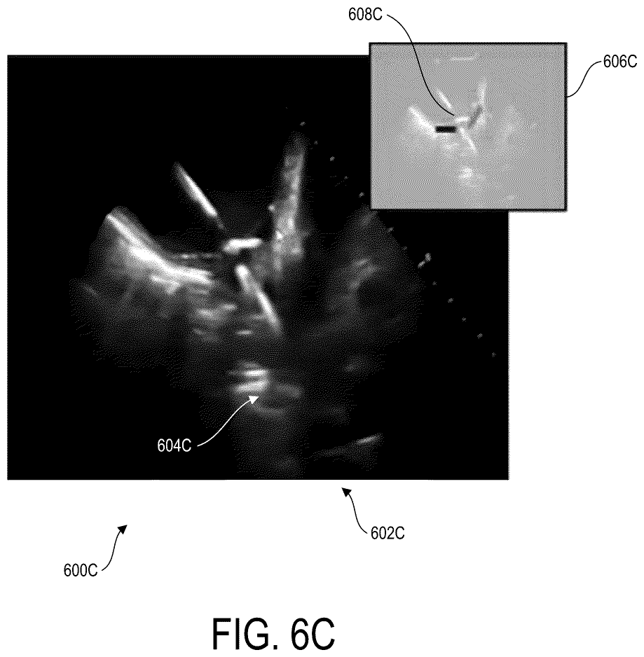

FIG. 6 illustrates an embodiment of a visualization 600 generated with the controller 400 operating process 500 and presented on a display device. The visualization 600 includes a two-dimensional ultrasound image 602. In the embodiment, the ultrasound image is in B-mode and includes anatomy 604 and an ablation catheter having a dynamic electrode assembly in the expanded configuration in view. The visualization 600 also includes a visual indicator 606 that provides information regarding the electrode assembly of the ablation catheter in the ultrasound image, such as an overlay with the ultrasound image 602. In the illustrated embodiment, the visual indicator 606 includes an electrode assembly element identifier 608. The electrode assembly element identifier 608 identifies elements of the electrode assembly, such as splines or electrodes, that are in view on the ultrasound image 602. In the illustrated example, the visual indicator 606 has delineated the splines of the electrode assembly in view on the ultrasound image 602, or in the imaging plane, with a color to identify the splines in view. In the illustrated embodiment, the electrode assembly includes five splines, and legend 610 identifying the splines and the relation of the splines to each other is in the visualization 600. The electrode assembly element identifier 608 has disposed a colored overlay on the splines in view as the blue spline and the yellow spline from the legend 610. In some embodiments, the legend 610 can include additional information regarding the splines, such as impedance information or proximity to tissue information. The visualization 600 permits the clinician to view the anatomy in B-mode while identifying which electrode assembly elements, such as splines, are in the imaging plane and the proximity of the splines to the anatomy.

The embodiment of the visualization 600 is a direct overlay on the ultrasound image 602 and the visual indicator 606 is disposed on the electrode assembly elements identified in the ultrasound image 602. FIGS. 6A and 6B illustrate embodiments visualizations 600A, 600B, respectively, also present direct overlays.

FIG. 6A illustrates visualization 600A having a two-dimensional ultrasound image 602A. In the embodiment, the ultrasound image is in B-mode and includes anatomy 604A and an ablation catheter having a dynamic electrode assembly in the expanded configuration in view. The visualization 600A also includes a visual indicator 606A that provides information regarding the electrode assembly of the ablation catheter in the ultrasound image, such as the direct overlay with the ultrasound image 602A. In the illustrated embodiment, the visual indicator 606A includes an electrode assembly element identifier 608A. The electrode assembly element identifier 608A identifies elements of the electrode assembly, such as splines, that are in view on the ultrasound image 602A. In the illustrated example, the visual indicator 606A has highlighted the splines of the electrode assembly in view on the ultrasound image 602A, or in the imaging plane, with a color to identify the splines in view.

FIG. 6B illustrates visualization 600B having a two-dimensional ultrasound image 602B. In the embodiment, the ultrasound image is in B-mode and includes anatomy 604B and an ablation catheter having a dynamic electrode assembly in the expanded configuration in view. The visualization 600B also includes a visual indicator 606B that provides information regarding the electrode assembly of the ablation catheter in the ultrasound image, such as the direct overlay with the ultrasound image 602B. In the illustrated embodiment, the visual indicator 606B includes an electrode assembly element identifier 608B. The electrode assembly element identifier 608B identifies elements of the electrode assembly, such as electrodes in the embodiment, that are in view on the ultrasound image 602B. In the illustrated example, the visual indicator 606B has highlighted the electrodes of the electrode assembly in view on the ultrasound image 602B, or in the imaging plane, with a color to identify the electrodes of the splines in view.

FIG. 6C illustrates a visualization 600C having an indirect overlay. The visualization 600C includes a two-dimensional ultrasound image 602C. In the embodiment, the ultrasound image is in B-mode and includes anatomy 604C and an ablation catheter having a dynamic electrode assembly in the expanded configuration in view. The visualization 600C also includes a visual indicator 606C that provides information regarding the electrode assembly of the ablation catheter in the ultrasound image, such as the indirect overlay, or inset, with the ultrasound image 602C. In the illustrated embodiment, the visual indicator 606C includes an electrode assembly element identifier 608C. The electrode assembly element identifier 608C identifies elements of the electrode assembly, such as the splines in the embodiment, that are in view on the ultrasound image 602C as in the inset 606C but does not obstruct the ultrasound image. In the illustrated example, the visual indicator 606C has highlighted the electrodes of the electrode assembly in view on the ultrasound image 602B, or in the imaging plane, with a color to identify the splines in view.

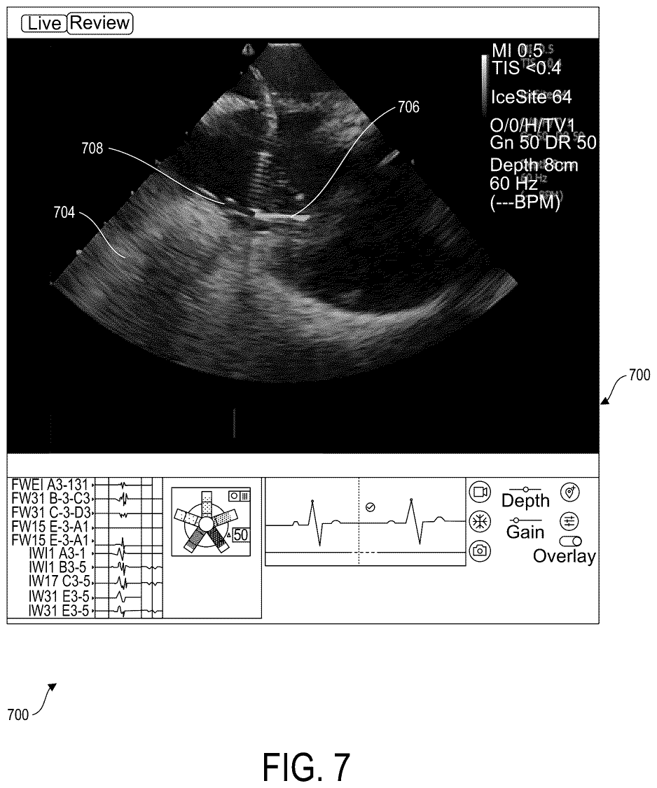

FIG. 7 illustrates an embodiment of a visualization 700 generated with the controller 400 operating process 500 and presented on a display device. The visualization 700 includes a two-dimensional ultrasound image 702. In the embodiment, the ultrasound image is in B-mode and includes anatomy 704 and an ablation catheter having a dynamic electrode assembly in the expanded configuration in view. The visualization 700 also includes a visual indicator 706 that provides information regarding the electrode assembly of the ablation catheter in the ultrasound image, such as an overlay with the ultrasound image 702. In the illustrated embodiment, the visual indicator 706 includes a contact visual identifier 708. The contact visual identifier 708 indicates whether a spline or electrode in the ultrasound image 702 is in contact with tissue. In the illustrated example, the visual indicator 706 has delineated the electrodes of the electrode assembly in contact with tissue on the ultrasound image 702, or in the imaging plane, with a color to identify the splines in view. The visualization 700 permits the clinician to view the anatomy in B-mode while identifying which electrodes are in the imaging plane and the whether the electrodes are in a predetermined proximity to, such as contact with, tissue. In visualization, one of splines includes an electrode in contact with tissue while the other of the splines does not, suggesting the clinician reposition the electrode assembly prior to activating an ablation pulse.

It is well understood that methods that include one or more steps, the order listed is not a limitation of the claim unless there are explicit or implicit statements to the contrary in the specification or claim itself. It is also well settled that the illustrated methods are just some examples of many examples disclosed, and certain steps may be added or omitted without departing from the scope of this disclosure. Such steps may include incorporating devices, systems, or methods or components thereof as well as what is well understood, routine, and conventional in the art.

The connecting lines shown in the various figures contained herein are intended to represent exemplary functional relationships and/or physical couplings between the various elements. It should be noted that many alternative or additional functional relationships or physical connections may be present in a practical system. However, the benefits, advantages, solutions to problems, and any elements that may cause any benefit, advantage, or solution to occur or become more pronounced are not to be construed as critical, required, or essential features or elements. The scope is accordingly to be limited by nothing other than the appended claims, in which reference to an element in the singular is not intended to mean “one and only one” unless explicitly so stated, but rather “one or more.” Moreover, where a phrase similar to “at least one of A, B, or C” is used in the claims, it is intended that the phrase be interpreted to mean that A alone may be present in an embodiment, B alone may be present in an embodiment, C alone may be present in an embodiment, or that any combination of the elements A, B or C may be present in a single embodiment; for example, A and B, A and C, B and C, or A and B and C. The terms “couples,” “coupled,” “connected,” “attached,” and the like along with variations thereof are used to include both arrangements wherein two or more components are in direct physical contact and arrangements wherein the two or more components are not in direct contact with each other (e.g., the components are “coupled” via at least a third component), but still cooperate or interact with each other.

In the detailed description herein, references to “one embodiment,” “an embodiment,” “an example embodiment,” etc., indicate that the embodiment described may include a particular feature, structure, or characteristic, but every embodiment may not necessarily include the particular feature, structure, or characteristic. Moreover, such phrases are not necessarily referring to the same embodiment. Further, when a particular feature, structure, or characteristic is described in connection with an embodiment, it is submitted that it is within the knowledge of one skilled in the art with the benefit of the present disclosure to affect such feature, structure, or characteristic in connection with other embodiments whether or not explicitly described. After reading the description, it will be apparent to one skilled in the relevant art(s) how to implement the disclosure in alternative embodiments.

Various modifications and additions can be made to the exemplary embodiments discussed without departing from the scope of the present disclosure. For example, while the embodiments described above refer to particular features, the scope of this disclosure also includes embodiments having different combinations of features and embodiments that do not include all of the described features. Accordingly, the scope of the present disclosure is intended to embrace all such alternatives, modifications, and variations as fall within the scope of the claims, together with all equivalents thereof.

We claim:

1. A medical imaging system, comprising:
a therapeutic, mapping or diagnostic catheter including a dynamic electrode assembly having a plurality of elements, the dynamic electrode assembly transitionable from a collapsed configuration to an expanded configuration;
an ultrasound imaging catheter configured to image the therapeutic, mapping or diagnostic catheter within a patient; and
a controller configured to:
generate a visualization for facilitation on a display, the visualization including an ultrasound image having a representation of an element of the electrode assembly based on signals provided by the ultrasound imaging catheter; and
identify the element of the electrode assembly from the representation of the element in the ultrasound image and position and orientation data obtained from the therapeutic, mapping or diagnostic catheter;
wherein the visualization further includes a visual indicator associated with the representation of the element based on the identified element of the electrode assembly.

2. The medical imaging system of claim 1, wherein the plurality of elements includes a plurality of splines and a plurality of electrodes.

3. The medical imaging system of claim 2, wherein the plurality of electrodes includes a plurality of ablation electrodes.

4. The medical imaging system of claim 1, wherein the visual indicator is an overlay on the ultrasound image.

5. The medical imaging system of claim 4, wherein the visual indicator is a direct overlay on the ultrasound image.

6. The medical imaging system of claim 5, wherein the direct overlay includes a delineation of the of at least one of a spline and an electrode.

7. The medical imaging system of claim 4, wherein the visual indicator is an indirect overlay.

8. The medical imaging system of claim 7, wherein the indirect overlay is an inset.

9. The medical imaging system of claim 1, wherein the controller configured to identify the element based on the three-dimensional position of the therapeutic, mapping or diagnostic catheter with respect to tissue as provided in an anatomical map.

10. The medical imaging system of claim 9, wherein the controller configured to identify the element is based on a detected shape of the electrode assembly.

11. The medical imaging system of claim 1, wherein the visualization includes a corresponding visual indicator in a representation of the element in a graphical representation generated via an electroanatomical mapping system.

12. The medical imaging system of claim 1, wherein the visual indicator indicates whether the identified element is within a predetermined proximity to tissue.

13. The medical imaging system of claim 12, wherein the visual indicator indicates whether the identified element is in contact with the tissue.

14. The medical imaging system of claim 1, wherein the therapeutic, mapping or diagnostic catheter is an electroporation catheter and the ultrasound imaging catheter is an intracardiac echocardiography (ICE) catheter.

15. A medical imaging controller, comprising:
a memory to store a set of instructions; and
a processor to execute the set of instructions to:
generate a visualization having an ultrasound image of a therapeutic, mapping or diagnostic catheter having an electrode assembly having a plurality of elements, wherein the ultrasound image includes a two-dimensional imaging plane having a representation of an element of the electrode assembly;
identify from the ultrasound image which element of the electrode assembly corresponds with the representation of the element of the electrode assembly based on position and orientation data obtained from the therapeutic, mapping or diagnostic catheter; and
generate a visual indicator in the visualization, the visual indicator associated with the identified element.

16. The medical imaging controller of claim 15, wherein the visual indicator indicates whether the identified element is within a predetermined proximity to tissue.

17. The medical imaging controller of claim 16, wherein the visual indicator indicates whether the identified element is in contact with the tissue.

18. The medical imaging controller of claim 15, wherein the controller configured to identify from the ultrasound image is based on a three-dimensional position of the ablation catheter with respect to tissue as provided in the anatomical map.

19. A method of generating an image for facilitation on a display, the method comprising:
generating a visualization having an ultrasound image of a therapeutic, mapping or diagnostic catheter having an electrode assembly having a plurality of elements, wherein the ultrasound image includes a two-dimensional imaging plane having a representation of an element of the electrode assembly;
identifying from the ultrasound image which element of the electrode assembly corresponds with the representation of the element of the electrode assembly base on position and orientation data obtained from the therapeutic, mapping or diagnostic catheter; and
generating a visual indicator in the visualization, the visual indicator associated with the identified element.

20. The method of claim 19 further comprising assigning an identifier to each element of the plurality of elements of the electrode assembly, and determining, based on the ultrasound image, which identifier corresponds with the representation of the element of the electrode assembly wherein the visual indicator is associated with the assigned identifier.
